(19) United States
(12) Patent Application Publication
(10) Pub. No.: US 2014/0377206 A1
(43) Pub. Date: Dec. 25, 2014

(54) SYSTEM USE OF SHAMPOO AND HAIR CONDITIONING COMPOSITION COMPRISING MONO-LONG ALKYL AMINE AND/OR POLYOL

(71) Applicant: THE PROCTER & GAMBLE COMPANY, CINCINNATI, OH (US)

(72) Inventors: Nobuaki UEHARA, Singapore (SG); Toshiyuki OKADA, Singapore (SG); Mui Siang SOH, Singapore (SG)

(21) Appl. No.: 14/308,856

(22) Filed: Jun. 19, 2014

Related U.S. Application Data
(60) Provisional application No. 61/836,673, filed on Jun. 19, 2013.

(51) Int. Cl. A61K 8/898; A61K 8/34; A61K 8/81; A61K 8/73; A61Q 5/12; A61Q 5/02

(52) U.S. Cl. CPC A61K 8/898; A61Q 5/12; A61Q 5/02; A61K 8/8158; A61K 8/731; A61K 8/737; A61K 8/345; A61K 2800/5426; A61K 2800/884
USPC 424/70.122

(57) ABSTRACT

Disclosed is a system of a hair shampoo composition and a hair conditioner composition, wherein the shampoo composition comprises a higher molecular weight and/or higher charge density cationic polymer, and wherein the conditioning composition comprises a cationic surfactant being an mono-long alkyl amine cationic surfactant and/or a polyol having a molecular weight of from about 40 to about 500. The system provides improved cleanness of the hair.

### FIELD OF THE INVENTION

The present invention relates to a system of a hair shampoo composition and a hair conditioner composition, wherein the shampoo composition comprises a higher molecular weight and/or higher charge density cationic polymer, and wherein the conditioning composition comprises a cationic surfactant being an mono-long alkyl amine cationic surfactant and/or a polyol having a molecular weight of from about 40 to about 500. The system provides improved cleanness of the hair.

### BACKGROUND OF THE INVENTION

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits.

Such conditioning agents are used in some shampoo compositions, as well as conditioning compositions.

However, there is a need for such shampoo compositions to provide improved cleanness, especially when used together with conditioning composition.

None of the existing art provides all of the advantages and benefits of the present invention.

### SUMMARY OF THE INVENTION

The present invention is directed to a system of a hair shampoo composition and a hair conditioner composition,

wherein the shampoo composition comprises: a detersive surfactant; a cationic polymer selected from the group of consisting of a high molecular weight cationic polymer having a molecular weight of from about 100,000 to about 5,000,000, a high charge density cationic polymers having a charge density of from about 0.5 to about 10.0, and mixtures thereof; and an aqueous carrier; and
wherein the conditioning composition comprises a cationic surfactant, and high melting point fatty compound, wherein:
(a) the cationic surfactant is an mono-long alkyl amine cationic surfactant, and the mono-long alkyl amine cationic surfactant is contained at a level by weight of the composition of from about 2% to about 25%; and/or
(b) the conditioning composition further comprises a polyol having a molecular weight of from about 40 to about 500.

The present invention provides improved cleanness such as less clumping of hair.

These and other features, aspects, and advantages of the present invention will become better understood from a reading of the following description, and appended claims.

### BRIEF DESCRIPTION OF THE DRAWINGS

FIG. 1 is a photo of cleanness evaluation results.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

### Shampoo-Conditioner System

By the system of a certain shampoo composition and a certain conditioner composition, which both are explained below in detail, the present invention provides improved cleanness. Cleanness includes, for example, less clumping of hair, less stickiness, less residual feel, less “weigh down” feel, less greasiness, clean look and feel, free flowing, and/or having a natural shine instead of greasy shine.

### Method of Use

The shampoo-conditioning system of the present invention is preferably used by a method comprising following steps:

(i) applying to a hair an effective amount of the shampoo composition, shampooing the hair, and washing out the shampoo composition from the hair;
(ii) applying to the hair an effective amount of the conditioning composition.

The method preferably further comprises the step (iii) rinsing the hair after applying the conditioning composition.

Effective amount herein is, for example, from about 0.1 ml to about 2 ml per 10 g of hair, preferably from about 0.2 ml to about 1.5 ml per 10 g of hair.

### Shampoo Composition

The shampoo composition comprises:

a detersive surfactant;
a cationic polymer selected from the group of consisting of a high molecular weight cationic polymer having a molecular weight of from about 100,000 to about 5,000,000, a high charge density cationic polymers having a charge density of from about 0.5 to about 10.0, and mixtures thereof; and
an aqueous carrier.

### Detersive Surfactant

The detersive surfactant may be selected from the group consisting of anionic detersive surfactants, zwitterionic or amphoteric detersive surfactants, and combinations thereof.

Preferred detersive surfactants are anionic surfactants. The concentration of the anionic surfactant component in the composition should be sufficient to provide the desired cleaning and lather performance, and generally range from about 5% to about 50%, preferably from about 8% to about 30%, more preferably from about 10% to about 25%, even more preferably from about 12% to about 22%.

Preferred anionic detersive surfactants for use in the compositions include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate, disodium laureth sulfosuccinate, disodium laureth-3 sulfosuccinates, dioctyl sodium sulfosuccinate, and combinations thereof.

In ethoxylated alkyl sulfate surfactants such as Sodium Lauryl Sulfate, it is preferred that the ethoxylation level is from 1-3 moles per molecule.

### Cationic Polymers

The cationic polymer can be included at a range of about 0.01% to about 10%, and more preferably from about 0.05% to about 5%, by weight of the shampoo composition.

The high molecular weight cationic polymer has an average molecular weight of from about 100,000 to about 5,000,000, preferably from about 400,000 to about 3,000,000, more preferably from about 800,000 to about 2,500,000.

The high charge density cationic polymers have a charge density of from about 0.1 meq/g to about 10 meq/g, preferably from about 0.7 meq/g to about 8.0 meq/g, more preferably from about 1.5 meq/g to about 7.0 meq/g.

Polymers having higher Mw and/or higher CD may be preferred in shampoo compositions in view of enhanced coacervate formation and/or increased substantivity on hair. However, it has been found by the inventors of the present invention that shampoos containing such polymers tend to have reduced clean feel after the hair is further treated by conditioning compositions. It has also been found by the inventors of the present invention that, by the system of such shampoo and the conditioning compositions of the present invention, clean feel can be improved.

The cationic polymer can be a naturally derived cationic polymer and/or synthetic cationic polymer. Representative examples and preferred examples of these polymers are shown below. Synthetic polymer may be preferred in view of improving its clean feel when the shampoo composition containing the same is used with the hair conditioning composition containing the polyol.

### Cationic Synthetic Polymer

Cationic synthetic polymer may be copolymers or homopolymers. In one embodiment, a homopolymer is utilized in the present composition. In another embodiment, a copolymer is utilized in the present composition. In another embodiment a mixture of a homopolymer and a copolymer is utilized in the present composition. In another embodiment, a homopolymer of a naturally derived nature, such as cellulose or guar polymer discussed herein, is combined with a homopolymer or copolymer of synthetic origin, such as those discussed below.

Homopolymers—Non-crosslinked cationic homopolymers of the following monomers are also useful herein: 3-acrylamidopropyltrimethylammonium chloride (APTAC), diallyldimethylammonium chloride (DADMAC), [(3-methylacrylolyamino)propyl]trimethylammonium chloride (MAPTAC), 3-methyl-1-vinylimidazolium chloride (QVI); [2-(acryloyloxy)ethyl]trimethylammonium chloride and [2-(acryloyloxy)propyl]trimethylammonium chloride.

Copolymers—copolymer may be comprises of two cationic monomer or a nonionic and cationic monomers.

Cationic synthetic polymers useful herein also include, for example, AM: Triquat copoylmers comprising: nonionic monomer unit being acrylamide (which can be referred as AM); and cationic monomer unit having the following formula (which can be referred as triquat):

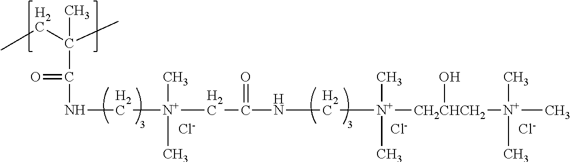

wherein the nonionic monomer is present in an amount from about 50% to about 99.5%, preferably from about 70% to about 99%, ore preferably from about 80% to about 99% by weight of the synthetic copolymer; and
wherein the cationic monomer portion is present in an amount from about 0.5% to about 50%, preferably from about 1% to about 30%, and more preferably from about 1% to about 20% by weight of the synthetic copolymer.

Non-limiting examples of suitable cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone.

Suitable cationic protonated amino and quaternary ammonium monomers, for inclusion in the cationic polymers of the composition herein, include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Other suitable cationic polymers for use in the compositions include copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16); copolymers of 1-vinyl-2-pyrrolidone and dimethylaminoethyl methacrylate (referred to in the industry by CTFA as Polyquaternium-11); cationic diallyl quaternary ammonium-containing polymers, including, for example, dimethyldiallylammonium chloride homopolymer, copolymers of acrylamide and dimethyldiallylammonium chloride (referred to in the industry by CTFA as Polyquaternium-6 and Polyquaternium-7, respectively); amphoteric copolymers of acrylic acid including copolymers of acrylic acid and dimethyldiallylammonium chloride (referred to in the industry by CTFA as Polyquaternium-22), terpolymers of acrylic acid with dimethyldiallylammonium chloride and acrylamide (referred to in the industry by CTFA as Polyquaternium-39), and terpolymers of acrylic acid with methacrylamidopropyl trimethylammonium chloride and methylacrylate (referred to in the industry by CTFA as Polyquaternium-47). Suitable cationic substituted monomers are the cationic substituted dialkylaminoalkyl acrylamides, dialkylaminoalkyl methacrylamides, and combinations thereof. These suitable monomers conform to the formula (IV):

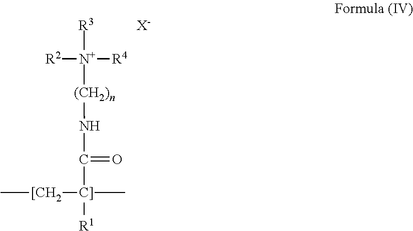

wherein R¹ of formula (IV) is hydrogen, methyl or ethyl; each of R², R³, and R⁴ of formula (IV) are independently hydrogen or a short chain alkyl having from about 1 to about 8 carbon atoms, typically from about 1 to about 5 carbon atoms, commonly from about 1 to about 2 carbon atoms; n of formula (IV) is an integer having a value of from about 1 to about 8, typically from about 1 to about 4; and X of formula (IV) is a counterion. The nitrogen attached to R², R³, and R⁴ of formula (IV) may be a protonated amine (primary, secondary, or tertiary), but is typically a quaternary ammonium wherein each of R², R³, and R⁴ of formula (IV) are alkyl groups, a non-limiting example of which is polymethyacrylamidopropyl trimonium chloride, available under the trade name POLYCARE® 133, from Rhone-Poulenc, Cranberry, N.J., U.S.A.

### Naturally Derived Cationic Polymer

Naturally derived cationic polymers for use in the composition include polysaccharide polymers, such as cationic cellulose derivatives and cationic starch derivatives. Suitable cationic polysaccharide polymers include those which conform to the formula (V):

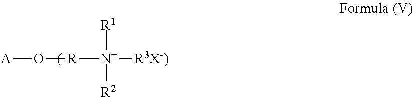

wherein A of formula (V) is an anhydroglucose residual group, such as a starch or cellulose anhydroglucose residual; R formula (V) is an alkylene oxyalkylene, polyoxyalkylene, or hydroxyalkylene group, or combination thereof; R¹, R², and R³ formula (V) independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R¹, R², and R³ formula (V)) typically being about 20 or less; and X formula (V) is an anionic counterion as described hereinbefore.

Other naturally derived cationic cellulose polymers are salts of hydroxyethyl cellulose reacted with trimethyl ammonium substituted epoxide, referred to in the industry (CTFA) as Polyquaternium 10 and available from Amerchol Corp. (Edison, N.J., USA) in their Polymer LR, JR, and KG series of polymers. Other suitable types of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp., under the tradename Polymer LM-200.

Other naturally derived cationic polymers include cationic galactomannan polymers such as cationic guar polymers and cationic cassia polymers. Cationic guar gum polymer include, for example, guar hydroxypropyltrimonium chloride, specific examples of which include the Jaguar series commercially available from Rhone-Poulenc Incorporated and the N-Hance series commercially available from Aqualon Division of Hercules, Inc, such as Nhance 3269, 3270, 3196.

Other naturally derived cationic polymers include quaternary nitrogen-containing cellulose ethers, some examples of which are described in U.S. Pat. No. 3,962,418. Other suitable cationic polymers include copolymers of etherified cellulose, guar and starch, some examples of which are described in U.S. Pat. No. 3,958,581.

When used, the cationic polymers herein are either soluble in the composition or are soluble in a complex coacervate phase in the composition formed by the cationic polymer and the detersive surfactant components described hereinbefore. Complex coacervates of the cationic polymer can also be formed with other charged materials in the composition.

### Aqueous Carrier for Shampoo

The shampoo composition comprises an aqueous carrier. The level and species of the carrier are selected according to the compatibility with other components and other desired characteristic of the product. Generally, the aqueous carrier is present in an amount from about 20% to about 95% by weight of the composition. An aqueous carrier may be selected such that the composition of the present invention may be in the form of, for example, a pourable liquid, a gel, a paste, a dried powder, or a dried film.

Aqueous carriers useful in the present invention include water and water solutions of lower alkyl alcohols. Lower alkyl alcohols useful herein are monohydric alcohols having 1 to 6 carbons, more preferably ethanol and isopropanol. The pH of the present composition, measured neat, is preferably from about 3 to about 9, more preferably from about 4 to about 8. Buffers and other pH-adjusting agents can be included to achieve the desirable pH.

### Additional Components for Shampoo

The shampoo composition of the present invention may further comprise one or more additional components known for use in hair care products, provided that the additional components are physically and chemically compatible with the essential components described herein, or do not otherwise unduly impair product stability, aesthetics or performance. Individual concentrations of such additional components may range from about 0.001% to about 10% by weight of the personal care compositions.

Non-limiting examples of additional components for use in the composition include conditioning agents (e.g., silicones, hydrocarbon oils, fatty esters), particles, anti-dandruff agents, suspending agents, paraffinic hydrocarbons, propellants, viscosity modifiers, dyes, non-volatile solvents or diluents (water-soluble and water-insoluble), pearlescent aids, foam boosters, additional surfactants or nonionic cosurfactants, pediculocides, pH adjusting agents, perfumes, preservatives, chelants, proteins, skin active agents, sunscreens, UV absorbers, and vitamins.

### Method of Making for Shampoo

The shampoo compositions may be made by mixing the ingredients together at either room temperature or at elevated temperature, e.g., about 72° C. Heat only needs to be used if solid ingredients are to be incorporated into the composition. The ingredients are mixed at the temperature. Additional ingredients, including electrolytes, polymers, fragrance, and particles, may be added to the product at room temperature.

### Hair Conditioning Composition

The hair conditioning composition comprises a cationic surfactant, and high melting point fatty compound, wherein:

(a) the cationic surfactant is an mono-long alkyl amine cationic surfactant, and the mono-long alkyl amine cationic surfactant is contained at a level by weight of the composition of from about 2% to about 25%; and/or
(b) the composition further comprises a polyol having a molecular weight of from about 40 to about 500.

The hair conditioning composition preferably further comprises aqueous carrier. Preferably, the cationic surfactants, the high melting point fatty compounds, polyol, and aqueous carrier are in the form of emulsion.

In the present invention, the total amount of the cationic surfactant and the high melting point fatty compound is preferably from about 4.5%, preferably from about 5.5%, more preferably from about 6.0% by weight of the composition, in view of providing the benefits of the present invention, and to about 50%, preferably to about 20%, preferably to about 17%, more preferably to about 15%, still more preferably to about 13% by weight of the composition, in view of product thickness, spreadability, dispensing and/or product appearance.

When increasing total amount of the cationic surfactant and the high melting point fatty compound, the composition may become: hard to spread; too thick; and/or hard to rinse. In such formulation, the benefit of the addition of polyol, and the benefit of the preparation method of the present invention may be more recognized.

The conditioning compositions of the present invention can be in the form of rinse-off products or leave-on products, and can be formulated in a wide variety of product forms, including but not limited to creams, gels, emulsions, mousses and sprays. The composition of the present invention is especially suitable for hair conditioners especially rinse-off hair conditioners.

### Cationic Surfactant

The cationic surfactant can be included in the composition at a level of from about 1.0%, preferably from about 1.5%, more preferably from about 2.0%, still more preferably from about 3.0%, and to about 25%, preferably to about 10%, more preferably to about 8.0%, still more preferably to about 6.0% by weight of the composition, in view of providing the benefits of the present invention.

Preferably, in the present invention, the surfactant is water-insoluble. In the present invention, “water-insoluble surfactants” means that the surfactants have a solubility in water at 25° C. of preferably below 0.5 g/100 g (excluding 0.5 g/100 g) water, more preferably 0.3 g/100 g water or less.

Cationic surfactant useful herein can be one cationic surfactant or a mixture of two or more cationic surfactants. Preferably, the cationic surfactant is selected from: mono-long alkyl quaternized ammonium salt; a combination of mono-long alkyl quaternized ammonium salt and di-long alkyl quaternized ammonium salt; mono-long alkyl amine; a combination of mono-long alkyl amine and di-long alkyl quaternized ammonium salt.

Cationic surfactant being a mono-long alkyl amine, more specifically, mono-long alkyl amidoamine may be preferred in view of improving its dry feel with the polyol.

Cationic surfactant being a mono-long alkyl quaternized ammonium salt may be preferred in view of improving its quick rinse feel with the polyol.

Cationic surfactant being either: a combination of mono-long alkyl quaternized ammonium salt and di-long alkyl quaternized ammonium salt; or a combination of mono-long alkyl amine and di-long alkyl quaternized ammonium salt, may be preferred in view of improving its dry feel such as less greasy and/or free flowing of hair (less clumping of hair), with the polyol.

### Mono-Long Alkyl Amine

Mono-long alkyl amine useful herein are those have one long alkyl chain of preferably from 12 to 30 carbon atoms, more preferably from 16 to 24 carbon atoms, still more preferably from 18 to 22 alkyl group. Mono-long alkyl amines useful herein also include mono-long alkyl amidoamines. Primary, secondary, and tertiary fatty amines are useful.

Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethyl amine, palmitamidopropyldimethylamine, palmitamidopropyldiethyl amine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al.

These amines are used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid, at a molar ratio of the amine to the acid of from about 1:0.3 to about 1:2, more preferably from about 1:0.4 to about 1:1.

### Mono-Long Alkyl Quaternized Ammonium Salt

The mono-long alkyl quaternized ammonium salts useful herein are those having one long alkyl chain which has from 12 to 30 carbon atoms, preferably from 16 to 24 carbon atoms, more preferably C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

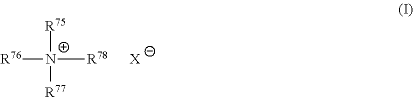

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

### Di-Long Alkyl Quaternized Ammonium Salts

When used, di-long alkyl quaternized ammonium salts are preferably combined with a mono-long alkyl quaternized ammonium salt or mono-long alkyl amine, at the weight ratio of from 1:1 to 1:5, more preferably from 1:1.2 to 1:5, still more preferably from 1:1.5 to 1:4, in view of stability in rheology and conditioning benefits.

Di-long alkyl quaternized ammonium salts useful herein are those having two long alkyl chains of from 12 to 30 carbon atoms, more preferably from 16 to 24 carbon atoms, still more preferably from 18 to 22 carbon atoms. Such di-long alkyl quaternized ammonium salts useful herein are those having the formula (I):

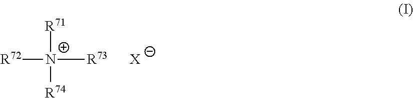

wherein two of R⁷¹, R⁷², R⁷³ and R⁷⁴ are selected from an aliphatic group of from 12 to 30 carbon atoms, preferably from 16 to 24 carbon atoms, more preferably from 18 to 22 carbon atoms or an aromatic, alkoxy, polyoxyalkylene, alkylamido, hydroxyalkyl, aryl or alkylaryl group having up to about 30 carbon atoms; the remainder of R⁷¹, R⁷², R⁷³ and R⁷⁴ are independently selected from an aliphatic group of from 1 to about 8 carbon atoms, preferably from 1 to 3 carbon atoms or an aromatic, alkoxy, polyoxyalkylene, alkylamido, hydroxyalkyl, aryl or alkylaryl group having up to about 8 carbon atoms; and X⁻ is a salt-forming anion selected from the group consisting of halides such as chloride and bromide, C1-C4 alkyl sulfate such as methosulfate and ethosulfate, and mixtures thereof. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Preferably, two of R⁷¹, R⁷², R⁷³ and R⁷⁴ are selected from an alkyl group of from 12 to 30 carbon atoms, preferably from 16 to 24 carbon atoms, more preferably from 18 to 22 carbon atoms; and the remainder of R⁷¹, R⁷², R⁷³ and R⁷⁴ are independently selected from CH₃, C₂H₅, C₂H₄OH, CH₂C₆H₅, and mixtures thereof.

Such preferred di-long alkyl cationic surfactants include, for example, dialkyl(14-18)dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

### High Melting Point Fatty Compound

The high melting point fatty compound can be included in the composition at a level of from about 2.5%, preferably from about 3.0%, more preferably from about 4.0%, still more preferably from about 5.0%, and to about 30%, preferably to about 10%, more preferably to about 8.0% by weight of the composition, in view of providing the benefits of the present invention.

The high melting point fatty compound useful herein have a melting point of 25° C. or higher, preferably 40° C. or higher, more preferably 45° C. or higher, still more preferably 50° C. or higher, in view of stability of the emulsion especially the gel matrix. Preferably, such melting point is up to about 90° C., more preferably up to about 80° C., still more preferably up to about 70° C., even more preferably up to about 65° C., in view of easier manufacturing and easier emulsification. In the present invention, the high melting point fatty compound can be used as a single compound or as a blend or mixture of at least two high melting point fatty compounds. When used as such blend or mixture, the above melting point means the melting point of the blend or mixture.

The high melting point fatty compound useful herein is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than the above preferred in the present invention. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols.

Preferred fatty alcohols include, for example, cetyl alcohol (having a melting point of about 56° C.), stearyl alcohol (having a melting point of about 58-59° C.), behenyl alcohol (having a melting point of about 71° C.), and mixtures thereof. These compounds are known to have the above melting point. However, they often have lower melting points when supplied, since such supplied products are often mixtures of fatty alcohols having alkyl chain length distribution in which the main alkyl chain is cetyl, stearyl or behenyl group.

In the present invention, more preferred fatty alcohol is a mixture of cetyl alcohol and stearyl alcohol.

Generally, in the mixture, the weight ratio of cetyl alcohol to stearyl alcohol is preferably from about 1:9 to about 9:1, more preferably from about 1:4 to about 4:1, still more preferably from about 1:2.3 to about 1.5:1 When using higher level of total cationic surfactant and high melting point fatty compounds, the mixture has the weight ratio of cetyl alcohol to stearyl alcohol of preferably from about 1:1 to about 4:1, more preferably from about 1:1 to about 2:1, still more preferably from about 1.2:1 to about 2:1, in view of avoiding to get too thick for spreadability. It may also provide more conditioning on damaged part of the hair.

### Polyol

Polyols can be included in the composition at a level of from about 0.5%, preferably from about 1.0%, more preferably from about 2.0%, still more preferably from about 3.0%, and to about 20%, preferably to about 15%, more preferably to about 10% by weight of the composition, in view of providing the benefits of the present invention.

Polyol useful herein are those having a molecular weight of from about 40 to about 500, preferably from about 50 to about 350, more preferably from about 50 to about 200, still more preferably from about 50 to about 150.

Preferably, polyols useful herein have from 2 to 12 OH groups, more preferably, 2-6, 8 or 10 OH groups, still more preferably 2-6 OH groups, even more preferably 2-4 OH groups.

Polyols useful herein are preferably water soluble. Water soluble polyols herein means those being soluble at a level used at 40° C. Non-water soluble polyols are, for example, glyceryl stearate.

Polyols useful herein include, for example: pentaerythritol; propylene glycol; butylene glycol; glycerin; pentylene glycol; hexylene glycol; Diols such as 1,2-diol, 1,3-diol, and other diols, the diols having a hydrocardon chain having 1-20 carbons, preferably 1-6 carbons; polyethylene glycol; polypropylene glycol; polybutylene glycol; polypentylene glycol; and polyhexylene glycol. Among them, preferred are Glycerin, Butylene Glycol, Propylene glycol, more preferred are glycerin.

### Aqueous Carrier for Conditioner

The composition of the present invention preferably comprises an aqueous carrier. The level and species of the carrier are selected according to the compatibility with other components, and other desired characteristic of the product.

The carrier useful in the present invention includes water and water solutions of lower alkyl alcohols. The lower alkyl alcohols useful herein are monohydric alcohols having 1 to 6 carbons, more preferably ethanol and isopropanol.

Preferably, the aqueous carrier is substantially water. Deionized water is preferably used. Water from natural sources including mineral cations can also be used, depending on the desired characteristic of the product. Generally, the compositions of the present invention comprise from about 0% to about 99%, preferably from about 50% to about 95%, and more preferably from about 70% to about 90%, and more preferably from about 80% to about 90% water.

### Gel Matrix

Preferably, in the hair conditioning composition, the emulsion is in the form of a gel matrix. The gel matrix comprises the cationic surfactant system, the high melting point fatty compound, and when contained, the polyol and an aqueous carrier. The gel matrix is suitable for providing various conditioning benefits, such as slippery feel during the application to wet hair and softness and moisturized feel on dry hair.

Preferably, when the gel matrix is formed, the cationic surfactant and the high melting point fatty compound are contained at a level such that the weight ratio of the cationic surfactant to the high melting point fatty compound is in the range of, preferably from about 1:1 to about 1:10, more preferably from about 1:1.5 to about 1:7, still more preferably from about 1:2 to about 1:6, in view of providing improved wet conditioning benefits.

Preferably, when the gel matrix is formed, the composition of the present invention is substantially free of anionic surfactants and anionic polymers, in view of stability of the gel matrix. In the present invention, “the composition being substantially free of anionic surfactants and anionic polymers” means that: the composition is free of anionic surfactants and anionic polymers; or, if the composition contains anionic surfactants and anionic polymers, the level of such anionic surfactants and anionic polymers is very low. In the present invention, a total level of such anionic surfactants and anionic polymers, if included, preferably 1% or less, more preferably 0.5% or less, still more preferably 0.1% or less by weight of the composition. Most preferably, the total level of such anionic surfactants and anionic polymers is 0% by weight of the composition.

### Silicone Compound

The conditioning compositions of the present invention may further contain a silicone compound. It is believed that the silicone compound can provide smoothness and softness on dry hair. The silicone compounds herein can be used at levels by weight of the composition of preferably from about 0.1% to about 20%, more preferably from about 0.5% to about 10%, still more preferably from about 1% to about 8%.

Preferably, the silicone compounds have an average particle size of from about 1 microns to about 50 microns, in the composition.

The silicone compounds useful herein, as a single compound, as a blend or mixture of at least two silicone compounds, or as a blend or mixture of at least one silicone compound and at least one solvent, have a viscosity of preferably from about 1,000 to about 2,000,000 mPa·s at 25° C.

The viscosity can be measured by means of a glass capillary viscometer as set forth in Dow Corning Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having conditioning properties can also be used.

Preferred polyalkyl siloxanes include, for example, polydimethylsiloxane, polydiethylsiloxane, and polymethylphenylsiloxane. Polydimethylsiloxane, which is also known as dimethicone, is especially preferred.

The above polyalkylsiloxanes are available, for example, as a mixture with silicone compounds having a lower viscosity. Such mixtures have a viscosity of preferably from about 1,000 mPa·s to about 100,000 mPa·s, more preferably from about 5,000 mPa·s to about 50,000 mPa·s. Such mixtures preferably comprise: (i) a first silicone having a viscosity of from about 100,000 mPa·s to about 30,000,000 mPa·s at 25° C., preferably from about 100,000 mPa·s to about 20,000,000 mPa·s; and (ii) a second silicone having a viscosity of from about 5 mPa·s to about 10,000 mPa·s at 25° C., preferably from about 5 mPa·s to about 5,000 mPa·s. Such mixtures useful herein include, for example, a blend of dimethicone having a viscosity of 18,000,000 mPa·s and dimethicone having a viscosity of 200 mPa·s available from GE Toshiba, and a blend of dimethicone having a viscosity of 18,000,000 mPa·s and cyclopentasiloxane available from GE Toshiba.

The silicone compounds useful herein also include a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. The silicone gums are available, for example, as a mixture with silicone compounds having a lower viscosity. Such mixtures useful herein include, for example, Gum/Cyclomethicone blend available from Shin-Etsu.

Silicone compounds useful herein also include amino substituted materials. Preferred aminosilicones include, for example, those which conform to the general formula (I):

(R₁)ₐG₃₋ₐ-Si—(—OSiG₂)ₙ-(—OSiG_{b}(R₁)_{2-b})ₘ—O—SiG₃₋ₐ(R₁)ₐ

wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A is a halide ion.

Highly preferred amino silicones are those corresponding to formula (I) wherein m=0, a=1, q=3, G=methyl, n is preferably from about 1500 to about 1700, more preferably about 1600; and L is —N(CH₃)₂ or —NH₂, more preferably —NH₂. Another highly preferred amino silicones are those corresponding to formula (I) wherein m=0, a=1, q=3, G=methyl, n is preferably from about 400 to about 600, more preferably about 500; and L is —N(CH₃)₂ or —NH₂, more preferably —NH₂. Such highly preferred amino silicones can be called as terminal aminosilicones, as one or both ends of the silicone chain are terminated by nitrogen containing group.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from the group consisting of non-polar, volatile hydrocarbons, volatile cyclic silicones, non-volatile linear silicones, and mixtures thereof. The non-volatile linear silicones useful herein are those having a viscosity of from about 1 to about 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s.

Other suitable alkylamino substituted silicone compounds include those having alkylamino substitutions as pendant groups of a silicone backbone. Highly preferred are those known as “amodimethicone”. Commercially available amodimethicones useful herein include, for example, BY16-872 available from Dow Corning.

The silicone compounds may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof.

### Additional Components for Conditioner

The conditioning composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; coloring agents, such as any of the FD&C or D&C dyes; perfumes; ultraviolet and infrared screening and absorbing agents such as benzophenones; and antidandruff agents such as zinc pyrithione, nonionic surfactant such as mono-9-octadecanoate poly(oxy-1,2-ethanediyl) supplied as, for example, Tween 20

### Method of Preparation for Conditioner

When the conditioning composition contains the polyol, it is preferred to prepare such composition by a method comprising a step of mixing the cationic surfactant system, high melting point fatty compound, and polyol to form an emulsion. When mixing, it is preferred to further contain an aqueous carrier.

It is believed that by this preparation method, the polyol is incorporated into emulsion structure, and thus, the composition provides improved benefits such as improved wet feel, improved dry feel, and/or improved stability, compared to the composition made by adding the polyol after the emulsion formed.

In view of incorporating more polyols into the emulsion, it is preferred to quickly form the emulsion by quickly cooling the mixture. Quickly cooling herein means 10° C./minute or more, 20° C./minute or more, 30° C./minute or more, 50° C./minute or more, 100° C./minute or more, 50° C./10 seconds or more.

### Examples

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

Shampoo compositions (wt %)

| | SH Ex. 1 | SH Ex. 2 | SH Ex. 3 |
|---|---|---|---|
| Water | q.s. | q.s. | q.s. |
| Cationic polymer-1 *1 | — | — | 0.25 |
| Cationic polymer-2 *2 | — | 0.25 | — |
| Cationic polymer-3 *3 | 0.5 | — | — |
| Sodium Laureth-3 Sulfate | — | 8.0 | 6.0 |
| (SLE3S) | | | |
| Sodium Lauryl Sulfate | — | 7.0 | 7.0 |
| (SLS) | | | |
| Ammonium Laureth-3 Sulfate | 7.5 | — | — |
| (ALE3S) | | | |
| Ammonium Lauryl Sulfate | 8.5 | — | — |
| (ALES) | | | |
| Cocoamidopropyl Betaine | — | 2.0 | 2.0 |
| Cocamide MEA | 0.8 | — | 0.85 |
| Sodium citrate | — | — | 0.88 |
| Sodium Chloride | — | 0.50 | 1.50 |
| Silicone microemulsion | — | 1.0 | — |
| PEG 7M | 0.1 | — | — |
| Dimethicone | 1.35 | — | 0.5 |
| Glycol Distearate | 1.5 | 1.5 | 1.5 |
| Fragrance | 0.5 | 0.80 | 0.50 |
| Preservatives, pH adjusters | Up to 1% | Up to 1% | Up to 1% |

Definitions of Components
*1 Cationic polymer-1: Copolymer of Acrylamide(AM) and TRIQUAT, having MW of 400,000 and CD of 1.6 meq/g, wherein AM:TRIQUAT ratio is 50:50
*2 Cationic polymer-2: Cationic guar having MW of 1,700,000 and CD of 0.7 meq/g
*3 Cationic polymer-3: Cationic cellulose having MW of 400,000 and CD of 0.7 meq/g

Conditioning compositions (wt %)

| | | CN | CN | CN | CN | CN |
|---|---|---|---|---|---|---|
| Components | | Ex. 1 | Ex. 2 | Ex. 3 | Ex. 4 | CEx. i |
| Group O | Behenyl | 2.97 | — | 2.97 | 2.22 | 2.97 |
| | trimethylammonium | | | | | |
| | methosulfate | | | | | |
| | Stearamidopropyl | — | 3.24 | — | — | — |
| | dimethyl amine | | | | | |
| | Dicetyl dimethyl | — | — | — | 0.74 | — |
| | ammonium chloride | | | | | |
| | Cetyl alcohol | 1.01 | 4.25 | 1.01 | 1.17 | 1.01 |
| | Stearyl alcohol | 2.53 | 2.93 | 2.53 | 2.94 | 2.53 |
| | Benzyl alcohol | 0.4 | 0.4 | 0.4 | 0.4 | 0.4 |
| Group W | Deionized Water | q.s. to 100% of the composition | | | | |
| | Polyol-1 *1 | 5 | 3 | — | 3 | — |
| | Polyol-2 *2 | — | — | 2 | — | — |
| | L-glutamic acid | — | 1.04 | — | — | — |
| | Preservative | 0.03 | 0.03 | 0.03 | 0.03 | 0.03 |
| Others | Aminosilicone *3 | 5 | — | — | — | 5 |
| | Perfume | 1.5 | 1.5 | 1.5 | 1.5 | 1.5 |
| | Panthenol | 0.5 | 0.5 | — | 0.5 | 0.5 |

Definitions of Components
*1 Polyol-1: Glycein
*2 Polyol-2: Butylene glycol
*3 Aminosilicone: Available from GE having a viscosity 10,000 mPa · s, and having following formula (I): (R₁)ₐG₃₋ₐ-Si—(—OSiG₂)ₙ-(—OSiG_{b}(R₁)_{2-b})ₘ-O—SiG₃₋ₐ(R₁)ₐ (I)

wherein G is methyl; a is an integer of 1; b is 0, 1 or 2, preferably 1; n is a number from 400 to about 600; m is an integer of 0; R₁ is a monovalent radical conforming to the general formula CqH_{2q}L, wherein q is an integer of 3 and L is —NH₂

### Method of Preparation

The above hair conditioning compositions of “Ex. 1” through “Ex. 4.” and “CEx. i” were prepared by the following method:

Group O components are mixed and heated to from about 66° C. to about 85° C. to form an oil phase. Separately, Group W components including the polyol are mixed and heated to from about 20° C. to about 48° C. to form an aqueous phase. In Becomix® direct injection rotor-stator homogenizer, the oil phase is injected and it takes 0.2 second or less for the oils phase to reach to a high shear field having an energy density of from 1.0×10⁵ J/m³ to 1.0×10⁷ J/m³ where the aqueous phase is already present. Other components are added to the gel matrix with agitation. Then the composition is cooled down to room temperature.

### Properties and Conditioning Benefits

For some of the above compositions, properties and benefits are evaluated by the following methods. Results of the evaluation are also shown below.

The embodiments disclosed and represented by “SH Ex. 1” through “SH Ex. 3” are hair shampoo compositions used in the system of the present invention, and the embodiments disclosed and represented by “CN Ex. 1” through “CN Ex. 4” are hair conditioning compositions used in the system of the present invention which are particularly useful for rinse-off use. In the system of the present invention, one of the above shampoo compositions is used in combination with one of the above conditioning compositions. Such systems have many advantages. For example, they provide improved cleanness.

Such advantages can be understood by the comparison between:

the system example of the present invention “SYS Ex. 1”, i.e., the system of the shampoo composition “SH Ex. 1” and the conditioning composition “CN Ex. 1”; and
the system comparative example “SYS CEx. i”, i.e., the system of the shampoo composition “SH Ex. 1” and the comparative conditioning composition “CN CEx. i”.

For example, improved cleanness such as less clumping of hair was observed in “SYS Ex. 1” of the present invention, compared to the comparative example “SYS CEx. i” which is almost identical to “SYS Ex. 1” except for using the conditioner composition which does not contain polyol.

### Cleanness

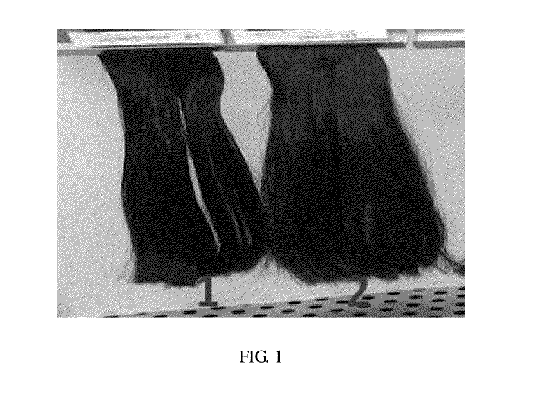

Cleanness is evaluated by visual comparison of the hair samples treated by the shampoo-conditioner systems. 20 g of Hair sample is wet using warm water. The hair sample is treated with shampoo (about 10% by weight of the hair), lathered and then the shampoo was rinsed off (about 20 seconds). The conditioner (about 10% by weight of the hair) is applied to the hair sample, and after being spread well, the conditioner is rinsed off (about 20 seconds). The hair samples were dried over night at room temperature before evaluated.

| | Shampoo | Conditioning | |
|---|---|---|---|
| System | composition | composition | Result |
| SYS Ex. 1 | SH Ex. 1 | CN Ex. 1 | FIG. 1 right side |
| | | | (marked as “2”) |
| SYS CEx. i | SH Ex. 1 | CN CEx. i | FIG. 1 left side |
| | | | (marked as “1”) |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A system of a hair shampoo composition and a hair conditioner composition,
wherein the shampoo composition comprises: a detersive surfactant; a cationic polymer selected from the group of consisting of a high molecular weight cationic polymer having a molecular weight of from about 100,000 to about 5,000,000, a high charge density cationic polymers having a charge density of from about 0.5 to about 10.0, and mixtures thereof; and an aqueous carrier; and
wherein the conditioning composition comprises a cationic surfactant, and high melting point fatty compound, wherein:
(a) the cationic surfactant is an mono-long alkyl amine cationic surfactant, and the mono-long alkyl amine cationic surfactant is contained at a level by weight of the composition of from about 2% to about 25%; and/or
(b) the conditioning composition further comprises a polyol having a molecular weight of from about 40 to about 500.

2. The system of claim 1, wherein the conditioning composition further comprises an aqueous carrier.

3. The system of claim 1, wherein the polyol is selected from the group consisting of glycerin, butylene glycol, propylene glycol, and mixtures thereof.

4. The system of claim 1, wherein the polyol is glycerin.

5. The system of claim 1, wherein the high molecular weight cationic polymer has an average molecular weight of from about 400,000 to about 3,000,000,

6. The system of claim 1, wherein the high molecular weight cationic polymer has an average molecular weight of from about 800,000 to about 2,500,000.

7. The system of claim 1, wherein the high charge density cationic polymers have a charge density of from about 0.7 meq/g to about 8.0 meq/g,

8. The system of claim 1, wherein the high charge density cationic polymers have a charge density of more preferably from about 1.5 meq/g to about 7.0 meq/g.
